(19) United States
(12) Patent Application Publication
(10) Pub. No.: US 2010/0063505 A1
(43) Pub. Date: Mar. 11, 2010

(54) BONE PLATE

(76) Inventors: Robert Frigg, Bettlach (CH); Alfred Niederberger, Salzburg (CH); Christoph Fuerst, Lohn-Ammannsegg (CH)

Correspondence Address: Fay Kaplun & Marcin, LLP, 150 Broadway, suite 702, New York, NY, 10038, US

(21) Appl. No.: 12/532,256

(22) PCT Filed: Mar. 22, 2007
(86) PCT No.: PCT/CH07/00157

§ 371 Date: Nov. 12, 2009

(51) Int. Cl. A61B 17/80

(52) U.S. Cl. 606/71

(57) ABSTRACT

A bone plate (1) with a longitudinal axis (2), a bottom surface (10) facing a bone and a top surface (11) and comprising a) an oblong base plate (3) with a anchoring portion (21) extending towards a first end (26) of the base plate (3) and a coupling portion (23) extending towards a second end (27) of the base plate (3); b) a sliding plate (6) connected with said coupling portion (23) which is slideable parallel to said longitudinal axis (2); whereby c) at least one first plate hole (4) is located in said anchoring portion (21) and at least one second plate hole (13) is located in said sliding plate (6); d) said base plate (3) and/or said sliding plate (6) is provided with retaining means (5) limiting a motion of said sliding plate (6) relative to said base plate (3) perpendicular to said top surface (11) in such manner that the sliding quality between said sliding plate (6) and said base plate (3) is maintained; and e) said at least one second plate hole (13) of said sliding plate (6) is provided with rigid locking means (12) allowing a rigid connection between a screw head (8) of a bone screw (7) insertable therein.

### FIELD OF THE INVENTION

The invention relates to a bone plate to be used for the treatment of fractured bones.

Presented in a simplified form the following subsequent processes can be differentiated from the fracture of a bone until the healing of the fracture:

1. the bone fractures;
2. the bone fracture is treated with a suitable implant;
3. at the fracture site the bone is initially degraded through osteoclasts;
4. and subsequently bone is constituted at the fractures site through osteoblasts; and
5. the fracture consolidates.

An essential requirement regarding the implant to be used consists in preventing relative motions between the bone fragments which could result in a resorption at the bone surfaces. If these interfragmentary disturbances remain (e.g. if no implant is set or else if a wrong implant is used) the resorptive degradation processes are more distinct than the stabilizing constitution processes. A gap remains between the fragments and the bony contact is not re-established. Such a pseudoarthrosis arises. If the screw heads of the bone screws are rigidly and angularly stable fixed in the bone plate and if the bone initially degrades at the fracture site it can happen that the two bone fragments loose the mutual connection. The bone plate thus keeps the two bone fragments at the same distance as before degradation. During this phase complications may also arise which are not inconsiderable. On the one hand the loading onto the implant is enhanced due to the lack of support through the bone. Thus, it is not rarely that a plate fracture at the height of the bone fracture in case of good bone quality occurs and that in case of a bad bone quality a screw can be pulled out from one of the bone fragments. On the other hand a too rigid fixation and the lack of a mechanical stimulation at the fracture site promotes the development of a pseudoarthrosis.

Depending on the stability of the splinting of the fracture the healing of the bone is different. It can be differentiated between the primary bone healing which mainly occurs in case of rigid fixation methods (implants) and the secondary bone healing which occurs in case of less rigid fixations methods (plaster bandage). In case of the first an immediate intracortical reformation of the bone occurs and the fracture is spanned without formation of callus. In case of the second the healing of the fracture occurs through formation of a callus at the fracture site, which converts in bone through gradual calcium dispersion. A faster healing of fractured bone with formation of callus has been observed by experiments with animals. At the same time the fractures have been exposed to cyclic compression. The result obtained was a significantly higher stability of the consoled fracture after several weeks.

Summarized, it can be stated that both a too high instability and a to high stability at the fracture site can result in a disturbance of the process of healing as far as in a pseudoarthrosis.

### DESCRIPTION OF THE PRIOR ART

A bone plate comprising a base plate and a sliding plate is known from U.S. Pat. No. 4,957,497 HOOGLAND. The disadvantage of this known device can be seen in the fact that the sliding plate is only laterally guided when fixed to the base plate. In case the bone screws are working loose in the bone the sliding plate could be dislocated from the base plate in a lateral direction resulting in a destabilization of the bone fixation device.

The German Utility Patent DE 20 2004 012 494 U MAIER discloses an osteosynthetic fixation device which is provided with a support plate and a second plate being slideably positioned relative to the support plate. A drawback of this fixation device can be seen in the fact that the two plates are pressed against each other by means of an additional fastener such that a frictional connection between the two plates results. By means of this frictional connection a further sliding between the support plate and the second plate is prevented once the complete plate is fixed at the bone.

### SUMMARY OF THE INVENTION

It is an object of the present invention to provide a bone plate allowing to mutually compress two bone fragments immediately after the bone fracture and to allow a limited axial displacement of the bone fragments relative to one another after bone resorption at the bone fragments at the fracture site has occurred. The invention solves the posed problem with a bone plate comprising a base plate with an anchoring portion extending towards a first end of the base plate and a coupling portion extending towards a second end of the base plate as well as with a sliding plate connected with said coupling portion which is slideable parallel to said longitudinal axis. In order to provide fixation means to fix the bone plate to a bone at least one first plate hole is located in said anchoring portion and at least one second plate hole is located in said sliding plate. Said base plate and/or said sliding plate is provided with retaining means limiting a motion of said sliding plate relative to said base plate perpendicular to the top surface of said base plate but not affecting the slideability between said sliding plate and said base plate. Further, said at least one second plate hole of said sliding plate is provided with rigid locking means allowing a rigid connection between the screw head of a bone screw insertable therein.

The essential advantages achieved by the invention are:
immediately after the bone fracture the two bone fragments can be mutually compressed; and
the gap subsequently occurring between the bone fragments through a resorption of the bone automatically closes by a limited displacement of the sliding plate relative to the base plate.

In one embodiment the rigid locking means are realised through at least one conical plate hole. The conical plate hole allows a rigid fixation of the screw head by maintaining the sliding quality between the two plates, i.e. the sliding plate would not be pressed against the base plate (neither towards the bottom surface nor towards to top surface) when the bone plate is fixed at the bone because once the conical screw head engages the conical plate hole the bone screw is simultaneously axially and rotatively locked and cannot be screwed deeper into the bone. The frictional engagement of the conical screw head and the conical plate hole acts as a stop.

Alternatively, the bone screw is screwed into the bone as far as the conical head contacts the conical plate hole and then the conical head is momentarily pressed against the sliding plate by means of an instrument. Thereby this instrument allows keeping a spacing between the sliding plate and the bone respectively between the base plate and the bone such allowing to maintain the slideable quality. After the frictional connection between the screw head and the plate hole is established the instrument is removed. Since cones used for a frictional connection usually have a taper below 5° the cone connection is self-locking and allows a rigid connection without the two parts being constantly pressed together.

Alternatively the rigid locking means may be realised by providing said at least one plate hole with a three-dimensional structure, e.g. in the form of an internal screw thread or a peripheral lamella or lip. These three-dimensional structures have the advantage to improve the rigidity of the connection between the screw head and the bone plate.

In a further embodiment the bone plate comprises additional sliding plates slideably arranged in the direction of the longitudinal axis on said base plate. The configuration of the bone plate with a plurality of sliding plates leads to a shorter contact surface between the sliding plates and the base plate so that a jamming of the sliding plates due to an elastic deformation caused by loads acting upon the bone plate can be prevented.

In a further embodiment the retaining means which limit a motion of said sliding plate relative to said base plate perpendicular to said longitudinal axis may comprise one of the following types of guidance between each other: dove tail guidance, groove type, rectangular form, curved form or rectangular form with concave lateral surfaces. By this guidance the effect of an elastic deformation of the sliding plate and/or the base plate is reduced such that the risk of a jamming of the sliding plate in the retaining means is also reduced.

In a further embodiment the base plate may have a complementary shape regarding to said sliding plate limiting (or preferably preventing) a motion of said sliding plate perpendicular to said longitudinal axis, whereby a higher stability of the bone plate is achieved by means of a lateral form fit

In a further embodiment at least one plate hole in the base plate may be configured in such a manner, that it allows a rigid connection with the head of a bone screw inserted therein. Due to this interlock between bone screw and bone plate, the bone plate must not be pressed onto the surface of the bone.

In a further embodiment the bone plate comprises second retaining means limiting the possible displacement of said sliding plate relative to said base plate parallel to said longitudinal axis and within a range x>0, Due to the limitation of the displacement of said sliding plate the surgeon may set the axially terminal bone screw in such manner that the shaft of the bone screw contacts the limitation so that the bone fractures abut on each other. Preferably, the sliding plate is displaceable within a distance of maximum 20 mm to 30 mm.

In a further embodiment said second retaining means comprise at least one elongate aperture which penetrates the base plate in the range of the sliding plate and which has a width “b” and a length “l” parallel to said longitudinal axis and wherein the passage of all plate holes arranged in said sliding plate are located within a distance “y” parallel to said longitudinal axis, said distance “y” being smaller than said length “l”.

In a further embodiment a clearance of maximum 1.0 mm, preferably maximum 0.1 mm is provided between the base plate and the sliding plate in a direction perpendicular to the top surface. A higher clearance reduces the risk of a jamming due to elastic deformation of the plates but allows a relative motion between the sliding plate and the base plate perpendicularly to the top surface. This relative motion between the sliding plate and the base plate perpendicularly to the top surface can lead to unsteadiness at the fracture site which may prevent the healing of the bone.

In a further embodiment the base plate has a one-piece configuration, which allows achieving a higher mechanical stability

In a further embodiment the base plate has a bottom surface apt for bone contact with an invariable geometry. Alternatively the sliding plate may be provided with a bottom surface not determined for bone contact.

In a further embodiment the base plate has an overall length L₀ and said coupling portion has a length L_{C}, whereby the ratio of the length L_{C} to the overall length L₀ is in the range of 25% to 60%. The base plate may have an overall length L₀ and said intermediate portion may have a length Lₗ, whereby the ratio of the length Lₗ to the overall length L₀ is preferably in the range of 5% to 30%. The resulting advantage is due to the fact that the stiffness of the bone plate in the range of the fracture site where the highest bending stress occurs is not weakened by the retaining means or plate holes.

In a further embodiment said rigid locking means are configured to prevent said sliding plate and said base plate from being pressed against each other such maintaining the slideability between said sliding plate and said base plate is not affected once the bone plate is fixed to a bone. By this measure the sliding plate can still slide smoothly and easily relative to the base plate.

The bone plate according to the invention may be used with at least one bone screw for the base plate and the sliding plate each.

In a further embodiment at least one of the bone screws comprises a screw head configured to match said rigid locking means such that upon tightening the bone screws no frictional connection between said sliding plate and said base plate is established. By this measure the sliding plate and the base plate are not pressed together when the bone screws are tightened. The sliding plate can still slide smoothly and easily relative to the base plate.

In a further embodiment at least one of the bone screws comprises a screw head configured to match said rigid locking means such that said sliding plate and said base plate are prevented from being pressed against each other allowing to maintain the slideability between the sliding plate and the base plate is not affected once the bone plate is fixed to a bone by means of bone screws.

### Brief Description of the Surgical Procedure:

The bone plate is set over the fracture line and anchored by means of locking head bone screws. The sliding plate can slide in the base plate until the first bone screw is inserted therein. To achieve a compression onto the fracture site one of the locking head bone screws is anchored in the bone through the sliding plate at the longitudinal end of the elongate aperture in the base plate. Thus, a longitudinal displacement of the sliding plate relative to the base plate is no longer possible.

### Bone Resorption:

Through bone resorption a gap occurs between the two bone fragments. Now, the sliding plate can longitudinally slide in the base plate. The dimension of this displacement is limited through the length of the elongate aperture in the base plate. The gap can be closed through the sliding possibility of the sliding plate. Further, it is possible to minimize these motions allowing controlled micro motions to occur such accelerating the consolidation of the bone fragments. The base plate and the sliding plate each are fixed to one of the bone fragments by means of at least two bone screws so that the displacement of the bone fragments relative to each other is guided through the displacement of the sliding plate in the direction of the longitudinal axis of the bone. Further, the bone screws cannot loosen due to the displacement of the bone fragments.

### A BRIEF DESCRIPTION OF THE DRAWINGS

Several embodiments of the invention will be described in the following by way of example and with reference to the accompanying drawings in which:

FIG. 1 illustrates a sectional view of an embodiment of the bone plate according to the invention;

FIG. 2 illustrates a top view onto the embodiment of FIG. 1;

FIG. 3 illustrates a front view of the embodiment of FIGS. 1 and 2 in the direction A;

FIG. 4 illustrates a perspective view of a further embodiment of the device according to the invention.

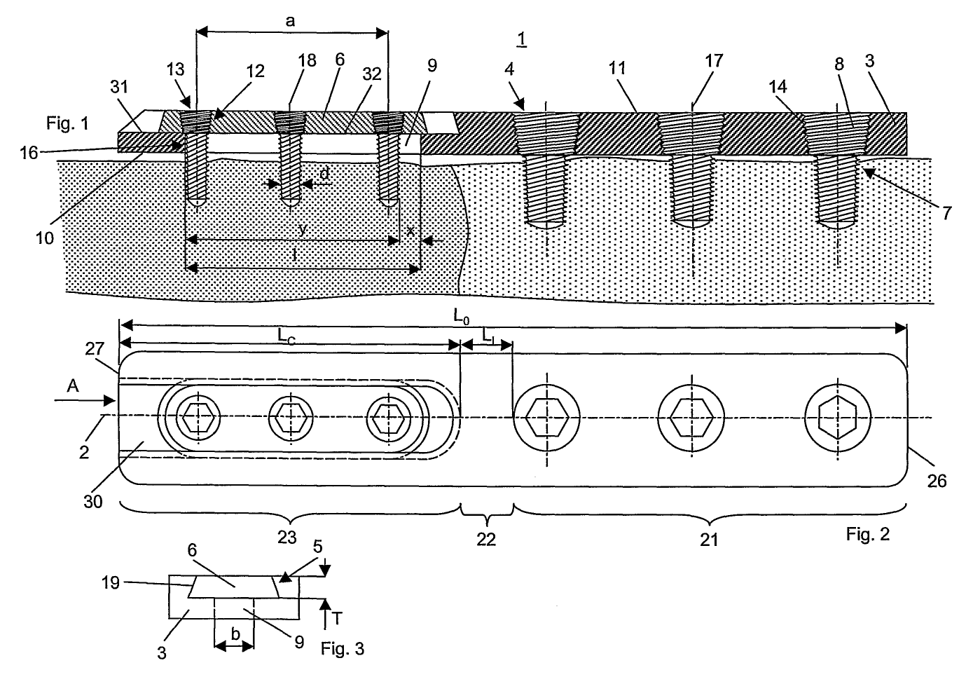

FIGS. 1 to 3 illustrate a bone plate 1 which comprises an oblong base plate 3 with an anchoring portion 21 extending towards a first end 26 of the base plate 3 and a coupling portion 23 extending towards a second end 27 of the base plate 3. Said anchoring portion 21 and said coupling portion 23 are arranged parallel to said longitudinal axis 2. Three first plate holes 4 are located in said anchoring portion 21 suitable for receiving bone screws 7. An intermediate portion 22 is provided which has no plate holes such that the bone plate 1 is not mechanically weakened at the fracture site 25. Further, in said coupling portion 23 of said base plate 3 a sliding plate 6 is arranged which has three second plate holes 13. Said base plate 3 has the shape of an oblong bar with an essentially rectangular cross sectional area perpendicular to said longitudinal axis 2, the two long sides of said cross sectional area coincide with the top surface 11, respectively the bottom surface 10 of said base plate 3. Each of said first plate holes 4 penetrates through said base plate 3 between said top surface 11 and said bottom surface 10 and has a first hole axis 17 cutting said longitudinal axis 2. Said sliding plate 6 is also bar-shaped and comprises three second plate holes 13 for receiving bone screws 7 (FIG. 1) and is arranged in a slideable manner parallel to the longitudinal axis 2 of said base plate 3.

As best seen in FIG. 3 the coupling of said sliding plate 6 to said base plate 3 is realised in such manner that the retaining means 5 which limit the motion of said sliding plate 6 relative to said base plate 3 perpendicular to the top surface 11 of said base plate 3 are realised by a dove tail guidance 19 wherein said sliding plate 6 is slideably accommodated. Said dove tail guidance 19 is configured as an elongated recess 30 in said base plate 3 which is open at the second end 27 of said base plate 3 and extends parallel to said longitudinal axis 2 on a length L_{C} of about 40% of the overall length L₀ of said base plate 3. Said elongated recess 30 penetrates into said base plate 3 from said top surface 11 to a depth T equal to the thickness of said sliding plate 6. The cross sectional area perpendicular to said longitudinal axis 2 of said elongated recess 30 linearly enlarges towards said bottom surface 10. At the depth T a supporting surface 31 extends parallel to said top surface 11 such that said sliding plate 6 is supported at its gliding surface 32 perpendicularly to said top surface 11. The cross section perpendicular to said longitudinal axis 2 of said sliding plate 6 has the form of an isosceles trapezium matching with the cross section of said elongated recess 30. The shape of said elongated recess 30 and of said sliding plate 6 such allow a sliding motion of said sliding plate 6 relative to said base plate 3 only and prevent motions of said sliding plate 6 relative to said base plate 3 perpendicular to said top surface 3 and laterally, i.e. perpendicular to the normal of the top surface 3 and in a direction perpendicular to said longitudinal axis 2. The so formed dove tail guidance 19 allows a precise displacement parallel to said longitudinal axis 2 of said sliding plate 6 relative to said base plate 3.

Further, each of said second plate holes 13 penetrates through said sliding plate 6 perpendicularly to said top surface 11 and has a second hole axis 18 cutting said longitudinal axis 2. In the range of said dove tail guidance 19 an elongate aperture 9 penetrating through said base plate 3 is provided. Said elongate aperture 9 has a width b measured perpendicular to said longitudinal axis 2 and a length l measured parallel to said longitudinal axis 2 such allowing the bone screws 7 inserted in said second plate holes 13 of said sliding plate 6 to pass through said base plate 3.

In this embodiment said first plate holes 4 in said base plate 3 and said second plate holes 13 in said sliding plate 6 are provided with rigid locking means 12. Each of said first and second plate holes 4,13 is conically configured and tapers towards the bottom surface 10 of said base plate 3, respectively the gliding surface 32 of said sliding plate 6. Further, each of said first and second plate holes 4, 13 is provided with a conical internal thread 14 such allowing a configuration of said rigid locking means 12 that include stopping means, i.e. a bone screw provided with a complementary conical threaded screw head 8 is fixed within the respective plate hole in a rigid, particularly angularly stable manner and further, the sliding plate 6 is prevented from being pressed against the base plate 3 (neither towards the bottom surface 10 nor towards to top surface 11) when the bone plate 1 is fixed at the bone 24 because once the threaded conical screw head 8 engages the respective conical plate hole the bone screw 7 is axially and rotatively locked and cannot be screwed deeper into the bone 24. Such said base plate 3 is rigidly kept at a distance to the central axis of the bone 24 and not necessarily contacts the surface of said bone 24 and said sliding plate 6 is rigidly kept at an appropriate distance to the central axis of the bone 24 as described above.

Additionally, the bone plate 1 comprises second retaining means 16 allowing to limit the sliding motion of said sliding plate 6 relative to said base plate 3. Said second retaining means 16 are realised through appropriately dimensioning the length l of said elongate aperture 9 in said base plate 3. Said length l exceeds the overall width y across the screw shafts of the two terminal bone screws 7 fixed in said second plate holes 13 of said sliding plate 6 such allowing an displaceability of said sliding plate 6 relative to said base plate 3 within a range x measured parallel to said longitudinal axis 2 (FIG. 1). Said overall width y is defined by distance a between the axes 18 of said terminal second plate holes 13 and the diameter d of the screw shaft of the bone screws 7 inserted in said second plate holes 13, i.e. y=a+d. Such the sliding motion of said sliding plate 6 parallel to said longitudinal axis 2 of the bone plate 1 is limited to said range x. FIG. 1 particularly illustrates the situation of the fracture treatment after the two bone fragments have been compressed when the bone plate 1 is fixed at the fractured bone 24.

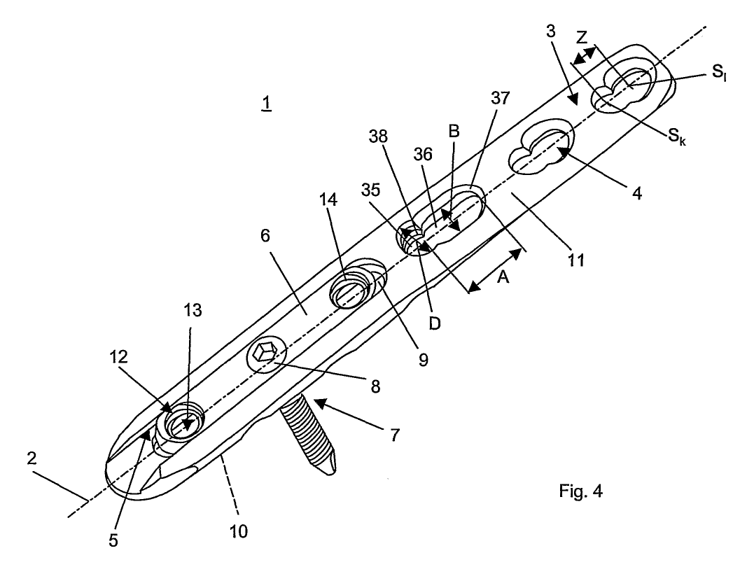

The embodiment illustrated in FIG. 4 differs from the embodiment shown in FIGS. 1-3 only therein, that the first plate holes 4 in the base plate 3 include two overlapping holes, a circular hole 35 with a diameter D and a centre of symmetry Sₖ, and an elongate hole 36 with a centre of symmetry Sₗ. The elongate hole 36 has a long axis A extending in the direction of the longitudinal axis 2 of said bone plate 1 and a short axis B extending vertically thereto. Further, the distance Z between the centres of symmetry Sₖ and Sₗ is smaller than the sum D/2+A/2. Both centres of symmetry are situated on the longitudinal axis 2 of said bone plate 1 whereby said circular hole 35 is axially terminally located such limiting said first plate hole 4 towards said sliding plate 6. In its upper portion, facing the top surface 11 of said bone plate 1, said elongate hole 36 has a concave, preferably spherical enlargement 37 for receiving a bone screw with a spherical screw head. Analogously to said first plate holes 4 according to the embodiment shown in FIGS. 1-3, said circular holes 35 each comprise rigid locking means 12 which are conically configured and have an internal screw thread 38 extending over the entire thickness of said base plate 3, from the top surface 11 to the bottom surface 10.

While various descriptions of the present invention are described above, it should be understood that the various features can be used singly or in any combination thereof. The scope of the present invention is accordingly defined as set forth in the appended claims.

1-21. (canceled)

22. A bone plate having a longitudinal axis, comprising:
a) an oblong base plate including an anchoring portion extending towards a first end of the base plate and a coupling portion extending towards a second end of the base plate;
b) a first sliding plate connected to the coupling portion so that it is slideable relative to the coupling portion parallel to the longitudinal axis; and
c) at least one first plate hole located in the anchoring portion and at least one second plate hole located in the sliding plate,
wherein at least one of the base plate and the first sliding plate includes a retaining arrangement limiting motion of the first sliding plate relative to the base plate in a direction perpendicular to a top surface of the bone plate while not impeding the sliding of the sliding plate relative to the base plate parallel to the longitudinal axis,
wherein the at least one second plate hole includes with a rigid locking arrangement allowing a rigid connection between a screw head of a bone screw insertable therein,
wherein the retaining arrangement includes an elongated recess penetrating into the base plate from the top surface to a depth T,
wherein, at the depth T, a supporting surface extends parallel to the top surface such that the first sliding plate is supported at the gliding surface perpendicularly to the top surface, and
wherein the bone plate includes a bottom surface which, when the bone plate is mounted on a bone in a desired configuration, faces the bone.

23. The bone plate according to claim 22, wherein the rigid locking arrangement includes at least one conical plate hole.

24. The bone plate according to claim 23, wherein the rigid locking arrangement is realized by providing the at least one plate hole with a three-dimensional structure including one of an internal screw thread, a peripheral lamella and a lip.

25. The bone plate according to claim 22, further comprising:
second and third sliding plates slideably coupled to the coupling portion and separated from one another and the first sliding plate along the longitudinal axis on the base plate.

26. The bone plate according to claim 22, wherein the retaining arrangement comprises one of a dove tail guidance, a groove type guidance mechanism, a rectangular form, a curved form and a rectangular form with concave lateral surfaces.

27. The bone plate according to claim 22, wherein the base plate has a complementary shape relative to the first sliding plate to limit motion of the first sliding plate perpendicular to the longitudinal axis.

28. The bone plate according to claim 27, wherein at least one plate hole in the base plate is configured in such manner that it allows a rigid connection with a head of the bone screw inserted therein.

29. The bone plate according to claim 22, further comprising:
a second retaining arrangement limiting displacement of the first sliding plate relative to the base plate parallel to the longitudinal axis within a range x>0,
wherein the second retaining arrangement includes at least one elongate aperture penetrating the base plate in a range of motion of the first sliding plate and having a width b and a length l parallel to the longitudinal axis, wherein all plate holes arranged in the first sliding plate are located within a distance y parallel to the longitudinal axis, the distance y being smaller than the length l.

30. The bone plate according to claim 22, wherein in a direction perpendicular to the top surface, a maximum clearance of 1.0 mm is provided between the base plate and the sliding plate.

31. The bone plate according to claim 30, wherein the maximum clearance is 0.1 mm.

32. The bone plate according to claim 22, wherein the base plate has a one-piece configuration.

33. The bone plate according to claim 22, wherein the bottom surface is adapted for bone contact with an invariable geometry.

34. The bone plate according to claim 22, wherein when base plate is coupled to a bone in a desired configuration, a bone facing surface of the first sliding plate does not contact the bone.

35. The bone plate according to claim 22, wherein the base plate has an overall length L0 and the coupling portion has a length LC, a ratio of the length LC to the overall length L0 being in a range of between 25% and 60%.

36. The bone plate according to claim 22, wherein the base plate has an overall length L0 and the intermediate portion has a length LI, a ratio of the length LI to the overall length L0 is in a range between 5% and 30%.

37. The bone plate according to claim 22, wherein the rigid locking arrangement prevents the first sliding plate and the base plate from being pressed against each other maintaining the slideability between the first sliding plate and the base plate after the bone plate has been fixed to a bone.

38. The bone plate according to claim 22, wherein the depth T is equal to a thickness of the first sliding plate.

39. The bone plate according to claim 22, wherein a cross section of the first sliding plate perpendicular to the longitudinal axis matches with a cross section of elongated recess.

40. A device, comprising:
a bone plate including a) an oblong base plate including an anchoring portion extending towards a first end of the base plate and a coupling portion extending towards a second end of the base plate; b) a sliding plate slidably connected to the coupling portion for sliding movement relative thereto parallel to the longitudinal axis; and c) at least one first plate hole located in the anchoring portion and at least one second plate hole located in the sliding plate; and
at least one bone screw for each of the base plate and the sliding plate,
wherein at least one of the base plate and the sliding plate includes a retaining arrangement limiting motion of the sliding plate relative to the base plate in a direction perpendicular to a top surface of the bone plate without impeding the sliding of the sliding plate relative to the base plate,
wherein the at least one second plate hole includes a rigid locking arrangement allowing a rigid connection between a screw head of a bone screw insertable therein,
wherein the retaining arrangement includes an elongated recess penetrating the base plate from the top surface to a depth T,
wherein, at the depth T, a supporting surface extends parallel to the top surface supporting the sliding plate thereon perpendicularly to the top surface, and
wherein the bone plate includes a bottom surface which, when the bone plate is mounted on a bone in a desired configuration, faces the bone.

41. The device according to claim 40, wherein the at least one bone screw includes a screw head configured to match the rigid locking arrangement such that upon tightening the bone screws, no frictional connection between the sliding plate and the base plate is established.

42. The device according to claim 40, wherein the at least one bone screw includes a screw head configured to match the rigid locking arrangement such that the sliding plate and the base plate are prevented from being pressed against each other and maintaining the slideability between the sliding plate and the base plate once the bone plate is fixed to a bone using bone screws.

43. The device according to claim 40, wherein the depth T is equal to a thickness of the sliding plate.

44. The device according to claim 40, wherein a cross section of the sliding plate perpendicular to the longitudinal axis matches a cross section of the elongated recess

45. A bone plate having a longitudinal axis, comprising:
a) an oblong base plate including an anchoring portion extending towards a first end of the base plate and a coupling portion extending towards a second end of the base plate;
b) a sliding plate slidably connected to the coupling portion for motion parallel to the longitudinal axis; and
c) at least one first plate hole located in the anchoring portion and at least one second plate hole located in the sliding plate, wherein the first plate hole is a combination hole;
wherein, at least one of the base plate and the sliding plate includes a retaining arrangement formed as an elongated recess extending into the base plate to a predetermined depth smaller than a depth of the base plate to limit movement of the sliding plate relative to the base plate in a direction perpendicular to a top surface of the bone plate while permitting slideable movement of the sliding plate parallel to the longitudinal axis, and
wherein, when the sliding plate is received in the retaining arrangement, a top surface of the sliding plate is parallel to the top surface of the base plate.

46. The bone plate of claim 45, wherein the combination hole includes first and second bores extending through the anchoring portion along hole axes separated from one another, the first and second bores being open to each other.

47. The bone plate of claim 45, wherein a cross-section of one of the first and second bores is substantially elliptical.
